(19) United States
(12) Patent Application Publication
(10) Pub. No.: US 2010/0113982 A1
(43) Pub. Date: May 6, 2010

(54) System for measurement and analysis of movement of anatomical joints and/or mechanical systems

(76) Inventor: Malcolm J. Smith, Ladera Ranch, CA (US)

Correspondence Address: Thomas J. Brindisi, Esq., 20 28th Place, Suite B, Venice, CA, 90291, US

(21) Appl. No.: 12/589,796

(22) Filed: Oct. 27, 2009

Related U.S. Application Data
(60) Provisional application No. 61/108,838, filed on Oct. 27, 2008.

(51) Int. Cl. A61B 5/11

(52) U.S. Cl. 600/595

(57) ABSTRACT

A system and method for measuring and analyzing movement of anatomical joints and/or mechanical systems, comprising one or more sensors capable of detecting human or animal joint movement or rotary or linear movement as created by sports, fitness or physical therapy machines and equipment; a sensor support system that enables the one or more sensors to be placed in contact with a human or animal joint or moving part or parts of a sports, fitness or physical therapy machine; and electronic circuitry and display means for analyzing and displaying information received from the one or more sensors.

### RELATED APPLICATIONS

This application claims the benefit of U.S. provisional patent application Ser. No. 61/108,838 filed Oct. 27, 2008 and entitled “A Wired or Wireless Real-time System Incorporating the use of Software, Firmware and Hardware to Measure the Degree of Movement of a Human or Animal Anatomical Joint, or the Degree of Movement of Any Mechanical Device as used in Physical fitness, Sports or Physical Therapy.” The disclosure of that provisional patent application is incorporated herein by reference as if fully set forth herein.

### FIELD OF THE INVENTION

The present invention relates to the fields of sports, sports medicine, physical fitness or physical therapy, and more particularly to a system for measuring and analyzing extension and/or flexion of anatomical joints, and/or rotary and/or linear movement of a mechanical system or machine.

### BACKGROUND

With the popularity of sports and the higher performance demands among athletes, people are pushing their bodies to their limits. This results in an increase in injuries. The most common type of injury usually occurs in the joints. Whether it is hyperextension, or tearing of tendons and ligaments, injury to the joints is fairly common. Extensive effort has been undertaken and various physical therapy programs developed in order to aid in the recovery of these injuries. One of the more difficult aspects of the recovery process is psychological, however; people who lose a certain degree of mobility often lose their desire to complete physical therapy, hampering and complicating the recovery process.

### SUMMARY OF THE INVENTION

The present invention comprises a system for measuring and analyzing movement (extension and/or flexion) of a human or animal anatomical joint, and/or the linear and/or rotational movement of a mechanical system, in conjunction with sports, physical fitness, or physical therapy. Sensors are attached externally to an anatomical joint, and/or to the moving parts of one or more mechanical systems. Information from the sensors is digitized, and software on a personal computer, PDA, embedded computer or cell phone is used to display, archive, compare, and analyze the sensor information.

The joint or machine movement information can be analyzed and responded to in real-time and/or archived for later comparison and analysis. Preferably, real-time range of movement information in the form of thermometer type gauges preferably is displayed, along with other information such as total weight moved, sets to-do and completed, repetitions to-do and completed, elapsed time for each component of the movement cycle (e.g., with a resolution down to one hundredth of a second), and/or other information relating to the movement cycle. Movement cycles preferably can be pre-defined and stored in a computer file system and recalled at any time.

In one embodiment of the invention, information associated with weight training and many other types of sports and fitness regimens can be monitored and logged. The system can instruct the user on which exercise to do next, the number of sets, repetitions, and the scheduled weight to use. Workout schedules can be pre-defined for any day of the week and month. The system saves complete information onto removable or non-removable storage devices for each exercise performed, so that trends and performance statistics can be reviewed at any time. This allows for comparison of an exercise performed today with that of an exercise completed a week ago, a month ago, or even years ago. The system displays real-time information so that the current exercise can be corrected while it is being performed, which helps ensure that exercises are performed correctly and safely. Preferably, the system can indicate to the user in real time if they are favoring one side of the body over the other, so the user can compensate with the weaker side of the body and ultimately reduce the tendency to over-compensate with the stronger side of the body.

### BRIEF DESCRIPTION OF THE DRAWINGS

FIG. 1 shows the sensor supports attached to a human elbow and knee extremity.

FIG. 2 shows the extremity elbow/knee textile support.

FIG. 3 shows the vacuum-sealed bend sensor.

FIG. 4 shows the transmitter and receiver system overview.

FIG. 5 shows the battery power management logic.

FIG. 6shows the personal computer software sensor calibration screen.

FIG. 7 shows the personal computer software main sensor information display screen.

FIG. 8 shows the personal computer software detail trending graphs.

FIG. 9 shows the personal computer software summary trending graphs.

FIG. 10 shows the personal computer database setup and configuration screen.

FIG. 11 shows the main exercise screen as displayed by a touch screen LCD display.

FIG. 12 shows another exercise screen.

FIG. 13 shows another exercise screen.

### DETAILED DESCRIPTION OF EMBODIMENTS

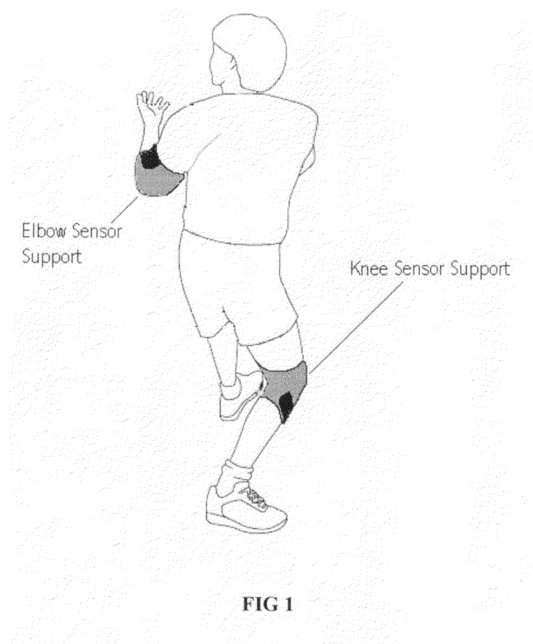

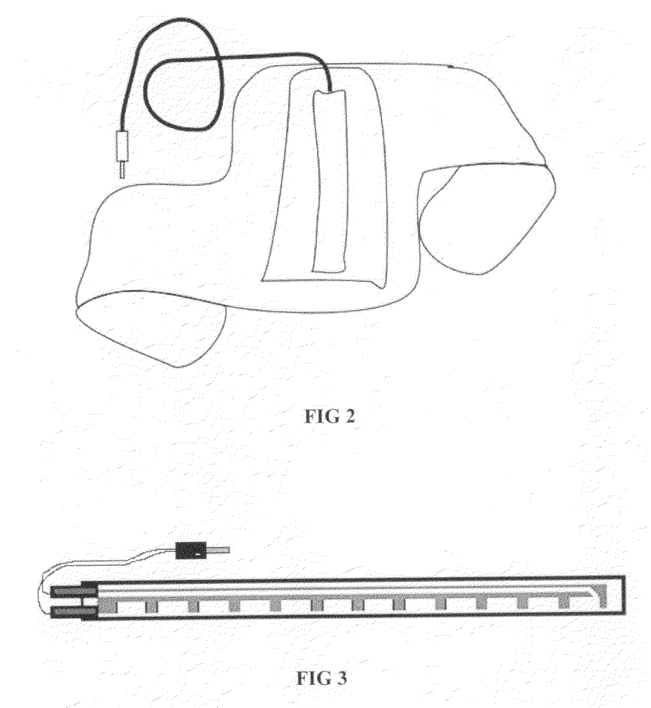

An embodiment of a system according to the invention in the context of an application relating to physical therapy, health, and/or fitness may include one or more sensors incorporated into aerated neoprene and nylon supports that are extremely adjustable, lightweight and comfortable, and worn on the body extremities as shown in FIG. 1. The sensors are secured and aligned to each neoprene and nylon support by means of an elastic layer that covers the sensor and holds it firmly against the support as shown in FIG. 2. The elastic prevents the sensor from moving out of alignment while range of motion remains unrestricted and unencumbered. The sensors are vacuum-sealed from the environment to eliminate dirt and moisture from entering the sensor and potentially damaging or affecting sensor signal integrity as shown in FIG. 3. Such a moisture-resistant system would accommodate the analysis of joints while exposed to, or submersed in water.

A sensor may utilize resistive characteristics that vary in response to an external physical force (such as a variable resister or strain gauge), or could be based on any other technology for measuring rotary or linear movement, such as optical incremental encoders, magnetic incremental encoders, potentiometers, bend-sensors.

The electrical sensors are secured externally to the body using textiles, hook and loop and plastics. The sensor attachments are comfortable, non-restrictive and aesthetically pleasing. Sensors are attached to the mechanical systems as appropriate and require a mechanical structure fabricated from ferrous or non-ferrous materials that ultimately allows the sensor to make contact with one or more moving parts of the target equipment. Possible points of contact for a sensor may be any one of the following, a pulley, gear, piston, cable or any moving part of the machine.

The sensor information and sensor processing electronics can be completely portable using radio frequency (RF) transmitter to carry the sensor information to the receiver unit, therefore the subject being monitored can be completely unrestricted and free to move in a natural manner. In some environments it may be advantageous to have the sensors wired to the processing electronics allowing the entire system to be connected to a standard AC power source thus eliminating the need for batteries. In addition to real-time user feedback, the system can store all movement information onto any digital storage device, including but not limited to USB flash-drive, SD card, SEEPROM or any magnetic media.

In the described embodiment, the sensor supports are primarily constructed of nylon or neoprene, but can be constructed of textiles, ferrous and no-ferrous metals, plastics, and/or any other materials suitable for securing the sensor to a joint or machine without impeding mobility or performance, with the size and type of materials used depending on the requirements of the joint to be analyzed.

Each sensor is connected to the signal processing electronics using fine gauge flexible electrical wire that has a small quick-disconnect jack on one end. This jack on each sensor connection allows the processing electronics to be removed from the subject being analyzed without the need to remove the sensors as well. Sensor connection wires can be placed under or clipped to clothing to eliminate the possibility of sensors being disconnected unintentionally.

### Sensor Signal Processing Flow

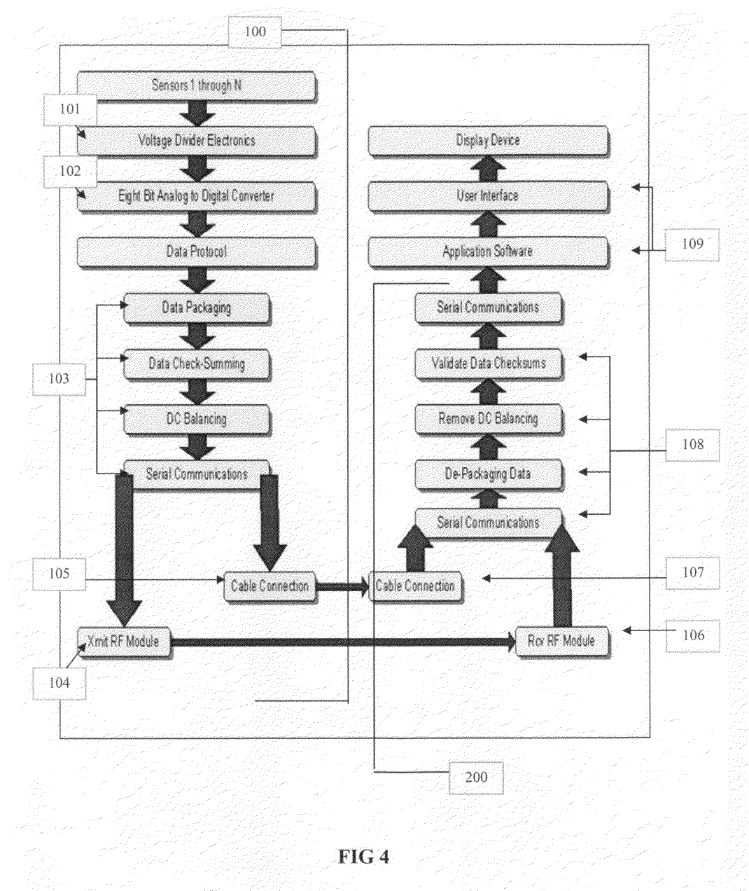

FIG. 4 shows the sensor signal processing flow for the Transmitter Unit 100 and the receiver 200. The system is a multiple stage system, where the first stage is a voltage divider circuit 101 that attaches directly to the sensors. The purpose of this stage is to convert the resistance from the sensor into an analog signal, with a signal in the range of 0-5 volts DC. The analog signal is in direct proportion with the amount of sensor flexion, in that the more the sensor is bent, the greater the analog signal outputs from stage one.

The second stage is the conversion of the analog signal into digital 102; this is accomplished using an eight-bit analog to digital converter (A/D Converter). Eight bits of resolution gives a digital range of zero through two fifty five decimal. The A/D converter circuitry (hardware) is incorporated in a microprocessor unit; the software that drives the A/D functionality was written by the inventor and will be discussed in detail later in this document.

The third stage is “data protocol” 103; this includes “Data Packaging,” “Data Check-summing,” “DC Balancing,” and “Serial Communications.” Data packaging is the process of prefixing and suffixing the signal data into a data packet. The system's data packet is very specific and is always the same number of eight bit bytes. The data packet incorporates three main components; (1) the packet header, this portion includes a unique identification number and other system information, (2) the sensor information for each of the sensors, (3) the packet trailer, this includes the data packet checksum. Data check-summing is a process where data can be validated by using a mathematical equation to ascertain if data was corrupted during transmission. For use in this system data check summing was accomplished by summing the original data bit with the interleaved DC balancing bit, (see paragraph (c) for more details). If the sum of the two bits is not equal to binary one then it is determined that data is corrupt and is discarded. DC balancing is process used by this system to better facilitate the transmission and reception of digital data using radio frequency (RF). DC balancing as used in this system ensures that no more than two binary bits of the same type is transmitted in sequence. This process is accomplished by adding an additional eight-bit byte to every data byte in the complete packet. The integration of the eight-bit byte is accomplished by interleaving a complimentary bit next to each bit of the original eight-bit byte. Serial Communications is a process where the finished data packet with DC Balancing and Check-summing is sent to the RF module for transmission.

The fourth stage is either the FCC compliant RF module (104) that transmits the data packets to the receiver module over a range of thirty meters or more, or a cable (105) connecting the transmitter with the receiver module. In either case the data is moved in serial mode from the transmitter to the receiver. All of the proceeding processes described in paragraphs a) through d) are performed in software that is executed on power-up of the transmitter module.

The fifth stage relates to either the FCC compliant RF module 106 that receives the data packets from the transmitter module, or a cable 107 connecting the receiver with the transmitter module. In either case the data is received in serial mode from the transmitter.

Stage six is the process of “De-Packaging Data” 108, “Remove DC Balancing,” “Validating Data Check Sums,” and “Serial Communications.” After data is received from the transmitter, the data must be “De-Packaged.” This is where the header is first examined to ensure that the unique ID is correct; if not then the entire data packet is discarded. If the unique ID is correct for this receiving pair then header and trailer portions of the data packet are stripped leaving only the DC Balanced sensor information. Next, the DC Balancing information must be removed by stripping every other binary bit from the data string. This data is then saved, as it is required to facilitate the check-summing phase. The data is then validated to ensure that it has not been corrupted. This is accomplished by summing each binary bit from each sensor reading and summing it to the associated previously preserved DC Balancing bit. If when summed the total is not equal to binary one, then the data byte is determined to be corrupt and is discarded. This process is repeated for each sensor reading until the entire data portion of the packet has been processed. Finally, the valid sensor readings are sent using serial (RS232 protocol) to the attached display unit.

The seventh stage relates to application software 109, which processes the received sensor information. The software is preferably adapted to facilitate the particular area of use without need for modifying hardware configurations.

### System Operation

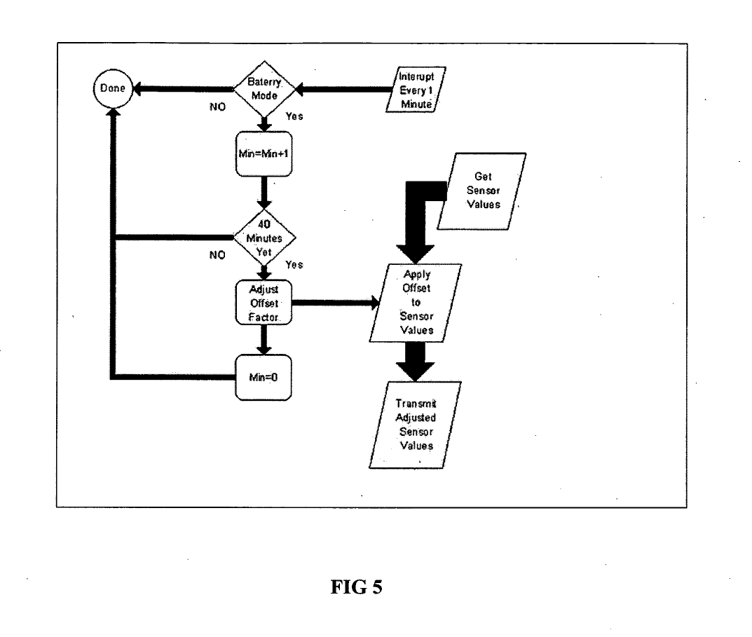

The transmitter module (not shown) is preferably located on or near the subject being analyzed, and has an external membrane keypad for user interaction. If the transmitter module is operating on battery power, then software (e.g., residing in flash memory within the transmitter module's microprocessor) routines monitor the battery condition as shown in FIG. 5. If the battery output is found to have decreased over a short period of time, (about forty minutes) the software re-calibrates the sensor signals to sustain consistent sensor output levels across the life of the battery. If the transmitter is connected to the receiver by wire, then power to the transmitter module can be derived from an AC to DC power supply connected to the receiver.

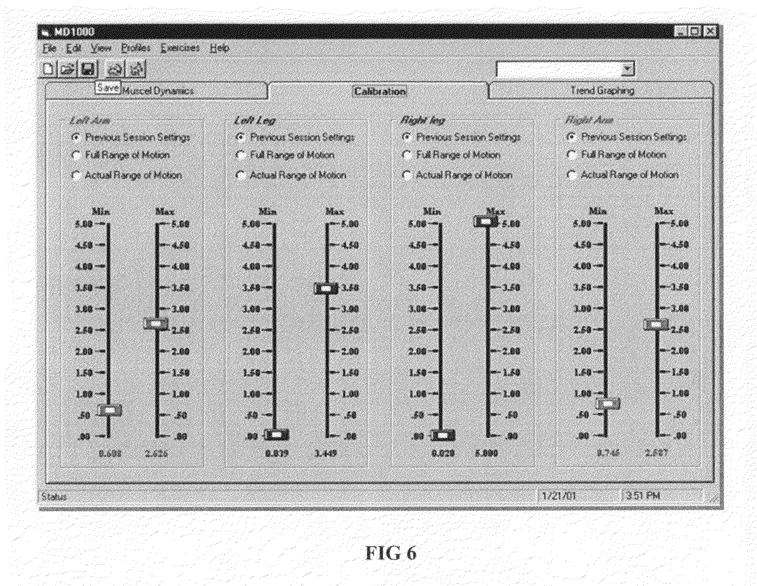

When the sensor supports are initially attached to the subject, the system must first be calibrated. This calibration is only required once at the start of any monitoring session. It is required that the subjects fully bend and straighten the joint being analyzed. (The calibration process may be manual or automatic; if automatic, the system preferably uses the first completed movement cycle to determine the begin and end range parameters). The software then sets the calibrated minimum and maximum for each joint calibrated. See FIG. 6 for software calibration screen. After calibration has been completed all data captured from the receiving module is buffered and the values adjusted based on the calibration factors. The calibration process makes it possible to show a full one hundred percent scale while the range on motion may be limited.

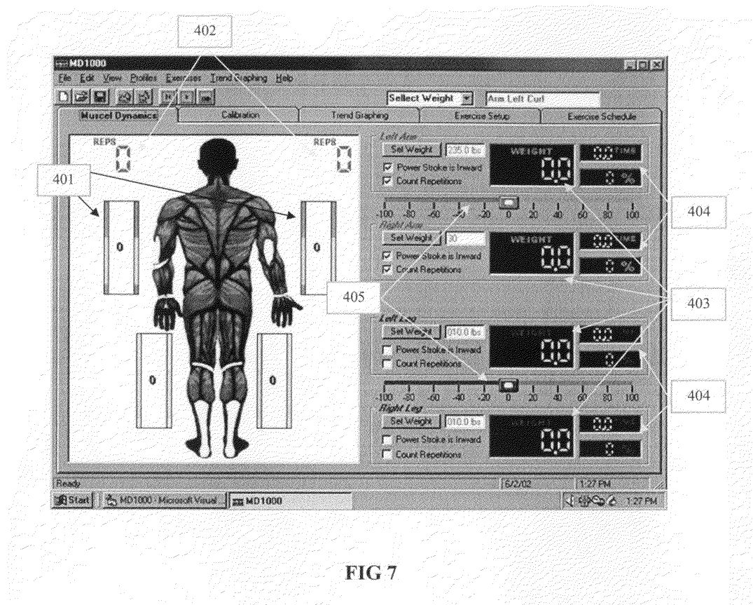

A thermometer type scale 401 is used to display movement activity in real time see FIG. 7. Digital counters are used to display total repetitions 402, total weight moved 403, and time elapsed for each completed repetition 404. The favor bar indicator 405 is used with parallel exercises when both joints (elbows or knees) are performing the same movements. The favor bar will move from its center position toward the left or right, to indicate the side (joint) that is ahead of the other. This occurs if the movements of both joints are not synchronized.

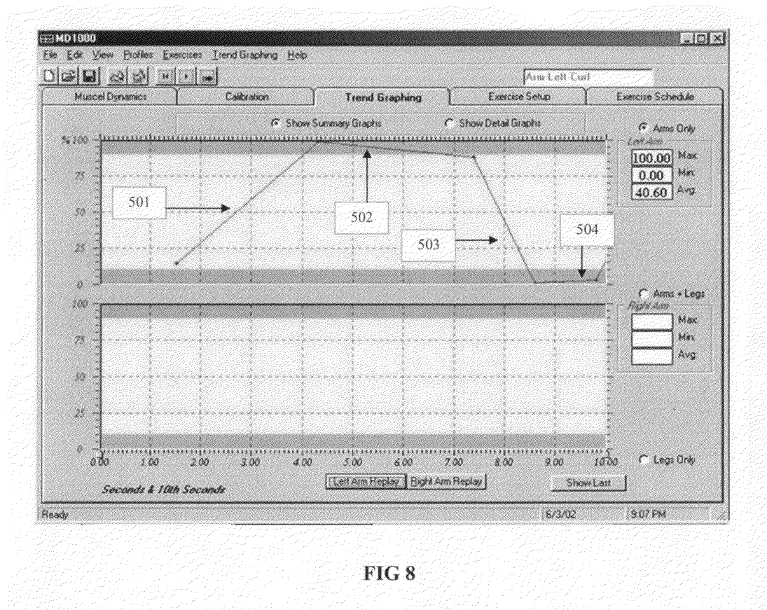

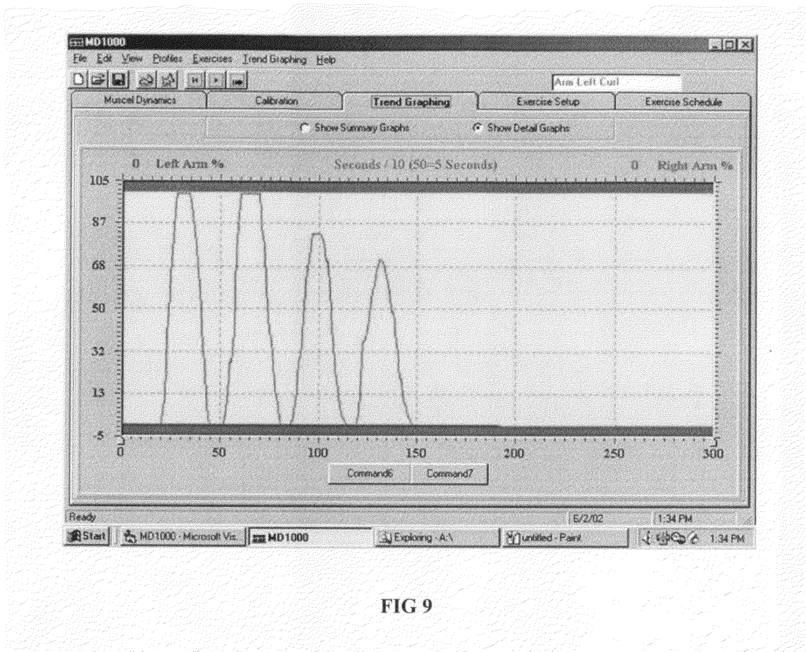

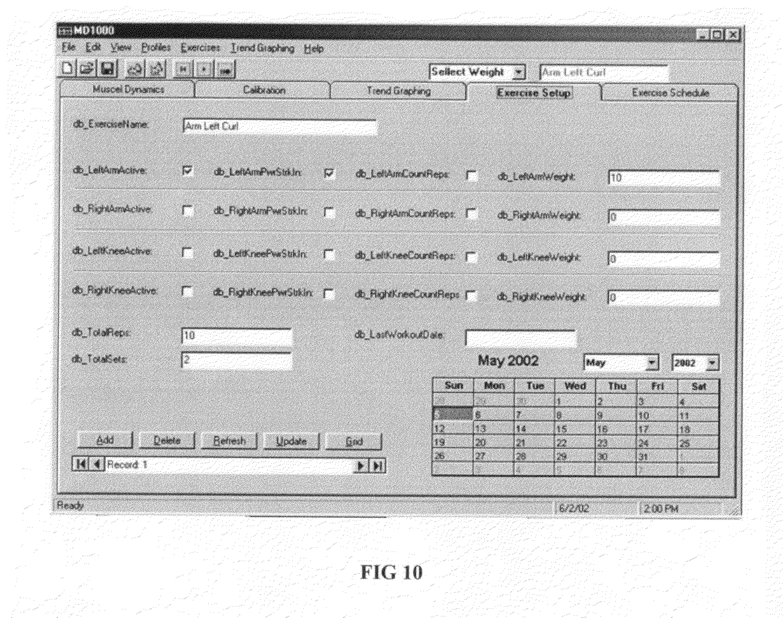

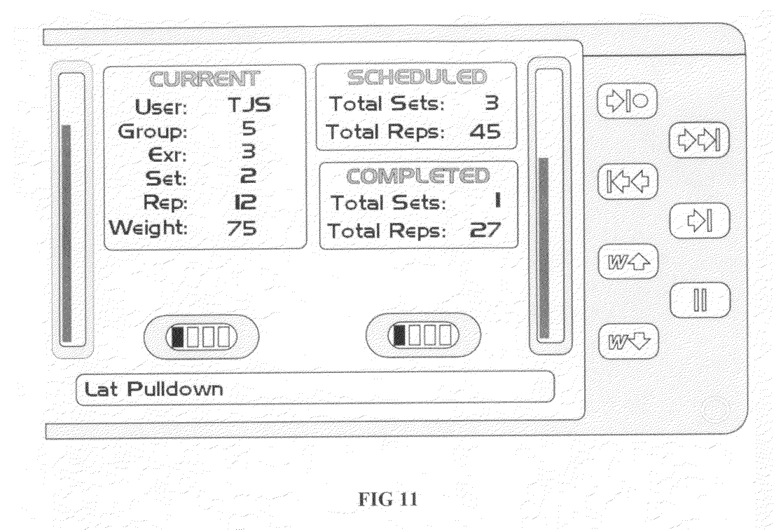

Joint movements can be compared with those completed hours, days, months, or years ago for complete trend analyses. See FIG. 8 for the detail-graphing screen. This graph shows the complete dynamics of the movement cycle. A typical movement cycle is comprised of four components; first a limb is moved away from its rest position to the eventual furthest extent of the movement 501. Second, a short pause with no movement 502. Third, the limb returns through the same path of travel to its original starting position 503. Fourth, a period of no movement (rest) 504, at this point the full movement cycle has been completed. See FIG. 9 for the trend summary, this graph better shows the performance for the movement set rather than the individual movement cycle. Movement profiles can be setup into a database for future retrieval and use. (See FIG. 10). An example of use would be for the health and fitness model where complete exercise regiments can be setup with total weight and the number of repetitions to be performed.

### Example of Use of the Described Embodiment

The end user first goes to a web site or runs application software that allows them to setup a workout schedule. This schedule includes the day of the week and the approximate time (AM or PM) that a workout will be performed. Next, the end user defines the actual exercises that they will be performing on the scheduled day and time. Exercise information may include the following, the exercise type, number of sets, number of repetitions per set, and the weight to use for each set. This information will be provided for as many individual exercises as required for any given day and time. After all exercise information for all scheduled days and times has been setup, then this information is saved onto the users USB flash-drive. When the user is ready to begin a workout, he/she simply inserts the USB flash-drive into the exercise machine(s) that has the system attached. After the USB flash-drive has been inserted, the system will display all defined workout schedules for the user and await selection. The system will then compare all of the user defined schedules with its internal real-time clock and find a schedule that best matches the current date and time and “rank” the schedules from best match at the top of the display to least best match at the bottom. Example: If the user has defined workout schedules for Monday, Wednesday, and Friday, and the current day of the week is Wednesday, then the system will rank the Wednesday schedule the highest and place it at the top of the selection screen. The user can simply accept that workout group or navigate to another in the group list if desired.

The system will now prompt the user to setup for the first exercise in the group. Setup information includes the following, the name of the exercise, where the user sits or stands with respect to the actual exercise machine, the total number of sets scheduled, the total number of repetitions for this set and the weight to use for this set. When the user is ready, he/she presses a footswitch or touches the touch-screen display to begin the exercise. The system will start recording information about the exercise and store it on the users USB flash-drive.

Real-time information is displayed on the television or touch screen LCD display for the user and consists of the following information, the current status of the exercise being performed for both the left and right as applicable, the number of repetitions completed, the number of sets completed, the number of sets remaining for this exercise, and the number of repetitions remaining for this set. As each exercise is being performed the dynamics of each repetition is recorded to the USB flash-drive. This information consists of recording four data points for each side (if applicable), 1-Start of the repetition, 2-Top of exercise, 3-Top and down and 4-Bottom of exercise. The time is recorded for each of the four data points with an accuracy of one hundredth of a second resolution.

After each exercise has been completed, the system advances to the next scheduled exercise automatically; however, the user can select any exercise and does not need to do the exercises in the same order as was originally defined. Regardless of what order exercises are completed, the system remembers all completed exercises and will indicate to the user that the “Workout is complete” only after each and every exercise in the current scheduled group has been completed. Additionally, the user can define messages that will be displayed by the system at any given point during the workout. Example, “Stretch for 10 minutes” or “15 minute cool-down.” After the scheduled exercise group has been completed, the user can (if desired) upload the captured information to a web site or a software application and then perform in-depth analysis of the captured data. Storing the current data in a database allows the user to compare current exercise performance information with that of a month or year ago etc.

The system can be connected to a phone line or the Internet where patient ‘at home therapy’ information can be sent directly to the clinic where it can be reviewed. After examining the information the therapists may prescribe additional exercises or increase the weight being used. This information could then be downloaded to the system and used the next time the exercise regiment is performed by the patient. This could possibly eliminate the need for patients to make frequent visits to a clinic for physical therapy treatments, and would be especially useful for patients who have difficulty getting to a clinic for therapy.

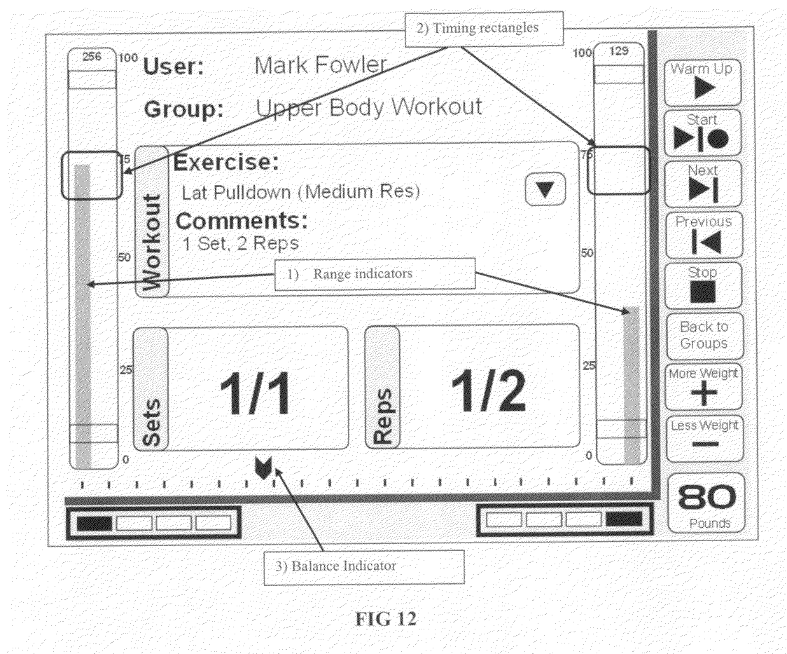

As shown in FIG. 12, Range Indicators (2) move up and down as the user performs the exercises. The example shows a lat pull down exercise being performed. The top of the range indicator within the scale (0 through 100%) reflects the current progress of the user's range continuously for both the power and return stroke. As this is a both arm-type exercise, both the left and right range indicators are displayed. The Timing Rectangles are animated blocks that move up when the user begins the power stroke, and then move down when the user begins the return stroke. The system is configured so that the rectangles move up and down at the optimal speed for each particular exercise, and the user then tries to keep the range indicators inside the rectangle while performing the exercise, facilitating optimal form and speed. The Balance Indicator (1) is displayed when a parallel exercise (i.e. both arms or both legs) is being performed. The goal is for the user to keep the balance indicator in the center of the display. The screen shot shows the indicator to the left side, because the user favored his left side (moving comparatively slower with his right arm). The Range Indicators (2) show that the right arm was behind the left. The Balance Indicator is particularly useful when adjusting for the natural urge to over-compensate with the body's stronger side. Having a user do a parallel exercise and then watching the Balance Indicator will immediately show if the user is right- or left-handed.

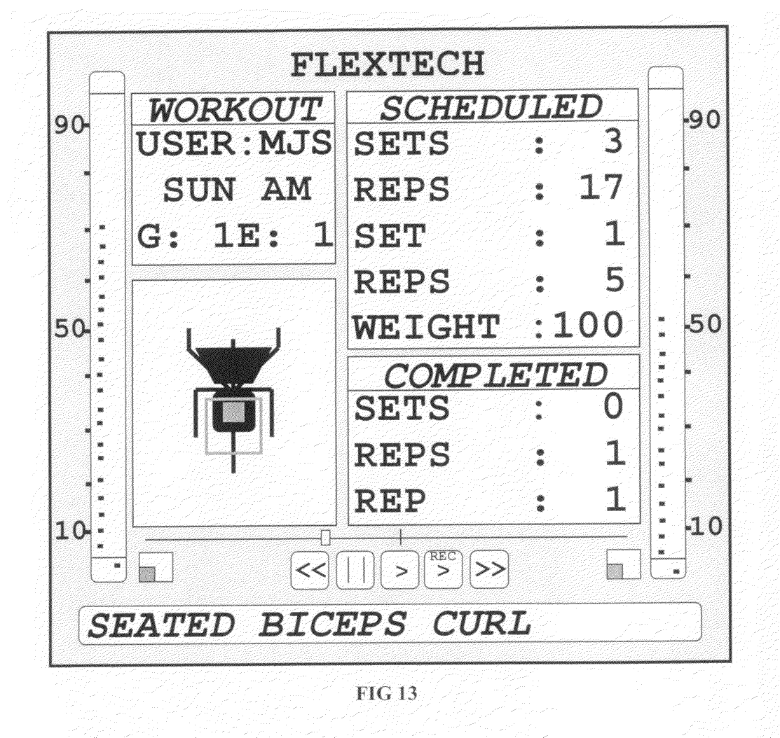

As shown in FIG. 13, an embodiment of the system may utilize dots to indicate to the user if they are performing the rep too fast or too slow. The dots are placed at regular time intervals at a location on the scale that indicates the percentage of range accomplished thus far for the repetition. If the rep is being performed too fast, the dots will lie farther apart; conversely, if the rep is being performed to slow, the dots will lie closer together. This allows a user to review performance while a rep is being performed, and also after it has been completed.

### Partial Element Listing

A SLAVE micro controller unit (MCU), micro processor unit (MPU) or central processing unit (CPU) that can communicate with the sensors, this slave system is used in a remote system when radio frequency is used to connect the sensors and slave processor with the host main processor in a wireless configuration. A collection of hardware support systems as required by the host MCU, MPU or CPU, these systems include SEEPROM, EEPROM, flash memory, RFID, Real-Time-Clock, USB Host and Slave systems, radio frequency (RF) systems, display systems including touch screen LCDs, LCDs, LEDs, NTSC devices, external switches including capacitive, resistive, mechanical, socket receptacles for sensors in and sensor out information and DC or AC power connections, power indicators and mechanical on/off or infer red (IR) type remote switch. A computer firmware application that resides within the instrument and is run by the host processor and provides for communications between the sensors and the host computer, generates all real-time information, displays the user options and saves all generated data to the digital storage device. The host computer can be an external personal computer, server or PDA or the host can be part of the instrument itself, in that it does not require any external computer system to monitor and process sensor signals, only a character or graphical color or monochrome display device is required to display real-time information for the user. A database setup and configuration screen used to store pre-defined exercises and other user information. An FCC compliant radio frequency transmitter and receiver working in conjunction to provide an interference free method of host slave communications at the office, hospital, doctors office or for any remote monitoring situation. An information processor and distributing device for viewing, analyzing, changing, reviewing, manipulating, monitoring, storing, backing-up, archiving and using for future use. Ports for wired connection of inputs, AC power, battery, printer, external memory, internet access, display screen, computer, etc. The user interface can be touch screen, voice recognition, or any mechanical or electrical switches.

### Various Preferred or Optional Features

Allow users to receive the most reliable real-time information allowing exercise users, patients and medical professionals to modify treatment plans based on the real-time information being displayed by software resulting from sensor monitoring. Adjust sensor resolution as appropriate for any given movement cycle, such as by the host sending commands to each sensor as determined by the exercise being performed. Memory stores the last exercise performed, so user(s) can resume workouts at any time. Allow doctors, clinicians, personal trainers, end users or any interested person to analyze, change, review, manipulate, monitor, save, back-up and archive data associated with exercises regimens and physical therapy sessions. Allow user defined or standard workout and physical therapy routines to be downloaded into the instrument either directly via the telephone line, internet or indirectly using a digital storage device. Allow end user to log-on to the software or firmware using a RFID card or tag, this could be a gym membership card or a wrist band or a key fob etc. Provide updated information by the clinician or personal trainer that can be downloaded for use the next time the exercise regiment is performed by the patient and or user. Permit information to be uploaded by the patient or exerciser to the doctor or personal trainer so that regiments can be analyzed and modified as needed. Allow the firmware in the host instrument to be field upgraded using a boot loader system. New firmware versions can be placed on a digital storage device such as USB flash drives, SD cards and inserted into the instrument or downloaded directly into the instrument over the internet or phone line. Can be completely stand-alone so as to be compact, portable, and cost effective. Multiple user profiles and information relating thereto can be input and manipulated.

The invention is not limited to human joints; in fact it can be used to monitor any anatomical joint, animal or human. For example, it could be used with racehorses recovering from knee surgery or other leg injuries. The sensors would be attached to both the injured and healthy leg and then the horse allowed to run freely in a paddock. The system then could remotely monitor the degree of flexion and range of motion of both the injured and non-injured leg. This information can then be examined to see if the injured leg has at least the same degree of movement as the healthy leg.

What is claimed is:

1. A system for measuring and analyzing movement of anatomical joints and/or mechanical systems, comprising:
a. one or more sensors capable of detecting human or animal joint movement or rotary or linear movement as created by sports, fitness or physical therapy machines and equipment;
b. a sensor support system that enables the one or more sensors to be placed in contact with a human or animal joint or moving part or parts of a sports, fitness or physical therapy machine; and
c. electronic circuitry and display means for analyzing and displaying information received from the one or more sensors.

2. A method of measuring and analyzing movement of anatomical joints and/or mechanical systems, comprising the following steps:
a. attaching one or more sensors to sensor supports;
b. attaching the one or more sensors and sensor supports to an anatomical joint or to a moving part of a mechanical system; and
c. connecting the one or more sensors to electronic circuitry and display means for analyzing and displaying information received from the one or more sensors.
